(19) United States
(12) Patent Application Publication
(10) Pub. No.: US 2025/0316286 A1
(43) Pub. Date: Oct. 9, 2025

(54) BIOFEEDBACK SYSTEM AND METHOD FOR SPEECH MODULATION

(71) Applicants: ARIZONA BOARD OF REGENTS ON BEHALF OF ARIZONA STATE UNIVERSITY, Scottsdale, AZ (US); DIGNITY HEALTH, SAN FRANCISCO, CA (US)

(72) Inventors: Nithish Kumar Saravanan, Tempe, AZ (US); Kirthik Roshan Nagaraj, Tempe, AZ (US); Ezhilan Veluchami, Tempe, AZ (US); Ramprakash Sridharan, Tempe, AZ (US); Tyler Hulson, Goodyear, AZ (US); Zane Reynolds, San Tan Valley, AZ (US); Tara Chay, Phoenix, AZ (US); Maura Rhodes, Mesa, AZ (US)

(21) Appl. No.: 19/170,356

(22) Filed: Apr. 4, 2025

Related U.S. Application Data
(60) Provisional application No. 63/574,771, filed on Apr. 4, 2024.

(51) Int. Cl. G10L 25/84; G10L 25/27

(52) U.S. Cl. CPC G10L 25/84; G10L 25/27

(57) ABSTRACT

Feedback systems and methods for speech modulation are provided. The feedback system includes a first microphone, a second microphone, a controller, and a biofeedback device. The first microphone captures a user's voice and the second microphone captures ambient noise. The controller processes inputs from the first microphone and the second microphone corresponding to the user's voice and the ambient noise, respectively, and provides a control signal based on the inputs. The biofeedback device receives the control signal from the controller to provide tactile feedback to the user, including a first form of tactile feedback indicating the user is speaking too softly and a second form of tactile feedback indicating the user is speaking too loudly.

### RELATED APPLICATIONS

This application claims priority under 35 U.S.C. § 119 to U.S. Provisional Patent Application No. 63/574,771 filed on Apr. 4, 2024, the entire contents of which is incorporated herein by reference.

### BACKGROUND

Motor, sensory, and/or cognitive impairments resulting from developmental or acquired disabilities can cause affected individuals to have difficulty modulating their vocal intensity (e.g., loudness). Parkinson's disease is the second most common neurodegenerative disorder in the United States, accounting for approximately one million people, with global prevalence estimates being close to ten million individuals. Additionally, individuals who have experienced brain injury, vocal fold paralysis/injury, or those with a variety of other conditions (e.g., Huntington's disease, cerebral palsy, etc.) have difficulty monitoring and effectively modulating vocal intensity. As such, millions of people worldwide grapple with the ability to speak clearly and, further, struggle to perceive and adjust their intensity independently.

These speech difficulties can greatly impact the quality of life for affected individuals. For example, speaking too softly and/or speaking too loudly in a given environment can impact social relationships and participation, resulting in isolation and reduced quality of life. While many individuals can implement strategies to achieve a more appropriate loudness level during skilled speech therapy, independent carry-over and self-monitoring of loudness levels in different environments after discharge from therapy can be difficult for these individuals. Accordingly, there is a need for systems and methods to assist individuals with speech disorders to provide guidance on adapting vocal intensity in real-life settings.

### SUMMARY

Some embodiments of the present disclosure may provide a feedback system for speech (e.g., voice) modulation of a user. In some embodiments, the feedback system comprises a first microphone to capture the user's voice, a second microphone to capture ambient noise, a controller, and a biofeedback device. In some embodiments, the feedback system comprises a low-pass filter to filter the inputs from the first microphone and the second microphone before they are processed by the controller. In some embodiments, the feedback system may comprise a user input that allows the user to adjust an intensity of the tactile feedback. In some embodiments, the feedback system may comprise a rechargeable power source that provides power to the controller via a power source management system and an on/off switch.

In some embodiments, the feedback system comprises a wearable garment. In some embodiments, the feedback system comprises a first microphone incorporated into the wearable garment to capture the user's voice. In some embodiments, the feedback system comprises a second microphone incorporated into the wearable garment to capture ambient noise. In some embodiments, the feedback system comprises a controller to process inputs from the first microphone and the second microphone corresponding to the user's voice and the ambient noise, respectively. In some embodiments, the feedback system comprises a biofeedback device, incorporated into the wearable garment, controlled by the controller to provide tactile feedback to the user based on the processed inputs.

In some embodiments, the wearable garment is a brace. In some embodiments, the brace is a shoulder brace. In some embodiments, the shoulder brace comprises a first pocket to receive a first housing comprising the first microphone, a second pocket to receive a second housing comprising the second microphone, and/or a third pocket to receive a third housing comprising the biofeedback device. In some embodiments, the shoulder brace comprises a first pocket to receive a first housing comprising the first microphone. In some embodiments, the shoulder brace comprises a second pocket to receive a second housing comprising the second microphone. In some embodiments, the shoulder brace comprises a third pocket to receive a third housing comprising the biofeedback device.

In some embodiments, the first housing, the second housing, and the third housing are removable from the first pocket, the second pocket, and the third pocket, respectively. In some embodiments, the first housing is removable from the first pocket. In some embodiments, the second housing is removable from the second pocket. In some embodiments, the third housing is removable from the third pocket. In some embodiments, the first housing, the second housing, and/or the third housing are connected via wired connections. In some embodiments, the wired connections between the first housing, the second housing, and/or the third housing are configured to be disconnected from each other.

In some embodiments, the controller may process inputs from the first microphone and the second microphone corresponding to the user's voice and the ambient noise, respectively, and provide a control signal based on the inputs.

In some embodiments, the first microphone is an adjustable gain microphone and the second microphone is an auto-gain microphone. In some embodiments, the first microphone is an adjustable gain microphone. In some embodiments, the second microphone is an auto-gain microphone.

In some embodiments, the biofeedback device may receive the control signal from the controller to provide tactile feedback to the user. In some embodiments, the biofeedback device may provide a first form of tactile feedback indicating the user is speaking too softly and a second form of tactile feedback indicating the user is speaking too loudly. In some embodiments, the first form of tactile feedback is continuous vibrotactile feedback and the second form of tactile feedback is intermittent vibrotactile feedback. In some embodiments, the first form of tactile feedback is continuous vibrotactile feedback. In some embodiments, the second form of tactile feedback is intermittent vibrotactile feedback.

In some embodiments, the wearable garment is one of a chest strap, an arm sleeve, an arm band, a leg sleeve, a leg band, a headband, a shirt, shorts, pants, a neckband, a ring, a patch, a watch, and a bracelet.

Some embodiments of the present disclosure may provide a method of providing feedback to a user for speech modulation. In some embodiments, the method comprises acquiring voice signals corresponding to a voice of the user, acquiring ambient signals corresponding to an ambient environment, processing the voice signals to determine a user's vocal state, processing the ambient signals to determine an ambient state, and providing tactile feedback to the user based on the user's vocal state relative to the ambient state. In some embodiments, the method further comprises determining that the user is speaking too softly when the user's vocal state is low. In some embodiments, the method further comprises determining that the user is speaking too softly when the user's vocal state is normal and the ambient state is noisy. In some embodiments, the method further comprises determining that the user is speaking too loudly when the user's vocal state is high, and the ambient state is not noisy.

In some embodiments, providing tactile feedback to the user based on the user's vocal state relative to the ambient state includes providing a first form of tactile feedback indicating the user is speaking too softly and providing a second form of tactile feedback indicating the user is speaking too loudly. In some embodiments, providing tactile feedback to the user based on the user's vocal state relative to the ambient state includes providing a form of tactile feedback indicating the user is speaking too softly. In some embodiments, providing tactile feedback to the user based on the user's vocal state relative to the ambient state includes providing a form of tactile feedback indicating the user is speaking too loudly. In some embodiments, providing the first form of tactile feedback indicating the user is speaking too softly includes providing continuous vibrotactile feedback and providing the second form of tactile feedback indicating the user is speaking too loudly includes providing intermittent vibrotactile feedback. In some embodiments, providing tactile feedback indicating the user is speaking too softly includes providing continuous vibrotactile feedback. In some embodiments, providing tactile feedback indicating the user is speaking too softly includes providing intermittent vibrotactile feedback.

In some embodiments, the user's vocal state is selected from a list comprising low, normal, and high, and the ambient state is selected from a list comprising noisy and not noisy. In some embodiments, the user's vocal state is selected from a list comprising low, normal, and high. In some embodiments, the ambient state is selected from a list comprising noisy and not noisy.

### DESCRIPTION OF THE DRAWINGS

FIG. 1 illustrates a schematic view of an example information processing model of a person with a neurodegenerative disorder.

FIG. 2 illustrates a schematic illustration of an example system according to some embodiments.

FIGS. 3A and 3B illustrate example plots (voltage versus time) of raw and filtered signals from microphones of the system in FIG. 2, where FIG. 3A illustrates a plot of signals from a user microphone and FIG. 3B illustrates a plot of signals from an ambient microphone.

FIG. 4 illustrates an electronic schematic diagram of another example system according to some embodiments.

FIG. 5 illustrates a perspective view of yet another example system according to some embodiments.

FIG. 6 illustrates a front view of the system of FIG. 5 incorporated into a wearable garment.

FIG. 7 illustrates a schematic front view of a user donning wearable garments that may incorporate the system of FIG. 5.

FIG. 8 illustrates an example sequence flow diagram, according to some embodiments, as a method for providing vibrotactile feedback to a user during speech.

FIG. 9 illustrates another example sequence flow diagram, according to some embodiments, as a method for providing vibrotactile feedback to a user during speech.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Although the present disclosure describes numerated embodiments, the embodiments described within each numerated embodiment may be combined or separated. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Generally, some embodiments provide a vibrotactile feedback system and method for assisting individuals with speech disorders, such as from neurodegenerative diseases. In some aspects, the system helps individuals adjust their vocal intensity based on the vibrotactile feedback. In additional aspects, the system incorporates microphones that collectively capture speech patterns and ambient sounds, and filtered signals from the microphones are interpreted by a responsive controller that generates a tailored vibrotactile feedback. This system can provide instant guidance to a use for adapting vocal intensity to ambient noise and also enables conversations without external intervention, incorporating a discreet design to ensure seamless integration into a user's daily life.

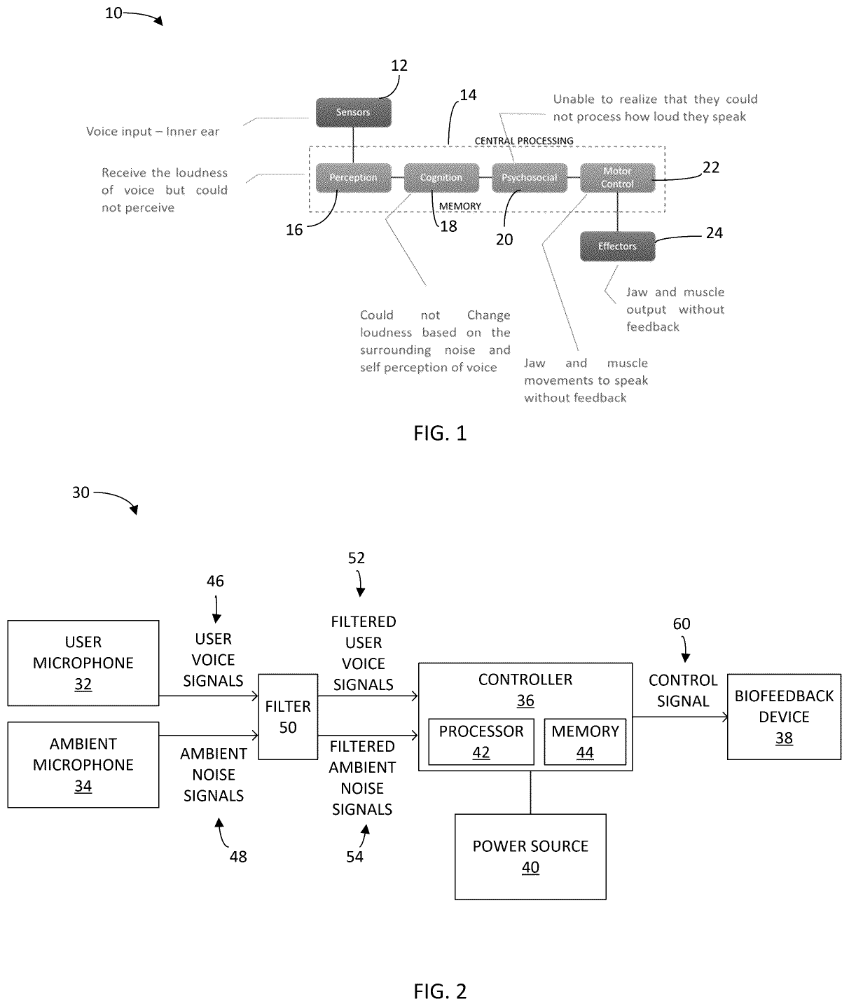

By way of example, FIG. 1 illustrates an information processing model 10 of a person with neurodegenerative disorder. The model includes sensors 12, a central processing unit 14 comprising a perception block 16, a cognition block 18, a psychosocial block 20, and a motor control block 22, and effectors 24. For a person with a speech disorder, such as Parkinson's disease, they may struggle to perceive and adjust their intensity independently. For example, their voice is sensed by the sensors 12, such as their ears. That input is received by the central processing unit 14 (e.g., the brain) and, at the perception block 16, the person receives the input corresponding to the loudness of their voice but may not be able to accurately perceive the loudness or relative loudness compared to the surrounding environment. Further, at the cognition block 18, the person may not have the self-awareness to modify the loudness of their voice based on the surrounding noise and self-perception of their voice. At the psychosocial block 20, the person may not realize the consequences on relationships and social interactions when they are unable to gauge how loudly they speak. Finally, at the motor control block 22, which can control effectors 24 such as the jaw and associated muscles, the motor control block 22 outputs instructions to the effectors 24 to continue speaking without proper feedback from the perception block 16, the cognition block 18, and/or the psychosocial block 20. As such, the effectors 24 operate (e.g., the person speaks) with inaccurate feedback from sensing their own voice.

Accordingly, individuals with neurodegenerative diseases or disorders may be considered to have a faulty internal feedback loop to assist with controlling their vocal intensity. However, external feedback can remarkably improve their performance. A first example of external feedback may be in the form of feedback from their conversation partners. However, while useful in therapy environments, direct external feedback may impair the flow of normal conversation and requires additional work by conversation partners, which may not be feasible or helpful in group situations.

Another example of external feedback includes a device that provides auditory feedback via an earpiece. The device is intended to increase the user's voice level through the Lombard effect (e.g., plays a babble sound cue inside the user's ear to trigger louder speech). The feedback provided to the user is locked at a specific intensity level that does not adjust or consider the environment (e.g. typically set in a quiet clinical environment absent of background noise) so the device cannot provide feedback to prompt the user to reduce their volume if it is too loud for a given ambient condition. In addition to the high price tag limiting its availability, the device is large and easily seen by others which might make users feel uncomfortable in social situations. Yet another example of external feedback includes a device worn on the wrist that provides visual feedback to the user. More specifically, the device is designed to activate a light when the user speaks with a suitable level of loudness. While the device may be generally affordable, it has a short life span, rendering it unsuitable for prolonged use. Furthermore, its conspicuous visibility to others could potentially compromise user privacy. The visual feedback occurring at the user's wrist may also unintentionally redirect an individual's focus toward the device, potentially diminishing interpersonal engagement and eye contact with others. Additionally, the device's inability to consider ambient noise levels for feedback might hinder its effectiveness in different environments.

The system of some embodiments, on the other hand, overcomes the shortcomings of these prior devices by not only combatting hypophonia (e.g., soft, quiet speech) by providing feedback when an individual is speaking too softly but, also, provides feedback when an individual is speaking too loudly and, further, considers ambient noise in its feedback loop. Generally, the system includes a biofeedback device that receives a control signal from a controller to provide tactile feedback to a user. The vibration feedback timing and pattern of the biofeedback is different under different scenarios, e.g., when the user is speaking too softly or too loudly. Further, the system of some embodiments can be discreet or invisible to conversation partners and provides feedback in a way that does not interfere with normal communication patterns.

For example, FIG. 2 illustrates a schematic illustration of an example biofeedback system 30 for speech modulation, according to some embodiments. The system 30 can include a first microphone 32 (e.g., a user microphone), a second microphone 34 (e.g., an ambient microphone), a controller 36, a biofeedback device 38, and a power source 40. Generally, the controller 36, powered by the power source 40, receives inputs from the first microphone 32 and the second microphone 34 and provides feedback to the user via the biofeedback device 38 to indicate whether the user is speaking too softly or too loudly. More specifically, the controller 36 can include a processor 42 and a memory 44 comprising data and instructions that, when executed by the processor 42, cause the processor 42 to perform certain functions, such as analyzing the inputs from the microphones 32, 34 and providing control signals to the biofeedback device 38. Accordingly, the system 30 can empower individuals to engage in more natural conversations by offering real-time guidance in modulating their vocal intensity.

As shown in FIG. 2, the system 30 includes two microphones 32, 34: the first microphone 32, e.g., a user microphone, can be used to capture the user's voice; and the second microphone 34, e.g., an ambient microphone, can be used to capture ambient noise. Accordingly, as further described below, the system 30 can consider both the user's vocal state and the ambient environment in determining whether to provide feedback to the user to increase or decrease their volume. In some embodiments, the user microphone 32 can be an adjustable gain microphone. As such, the gain of the microphone 32 can be optimized so that it captures the user's voice and eliminates or minimizes ambient noise. Though the gain is optimized, the user microphone 32 may still capture some background noise, but it may be considered negligible. Additionally, in some embodiments, the ambient microphone 34 can be an auto-gain microphone. The gain of the ambient microphone 34 can be adjusted automatically and can be directly proportional to the distance of the sound source. As such, the ambient microphone 34 can be effective at capturing background noise, including background noise that is relatively far away.

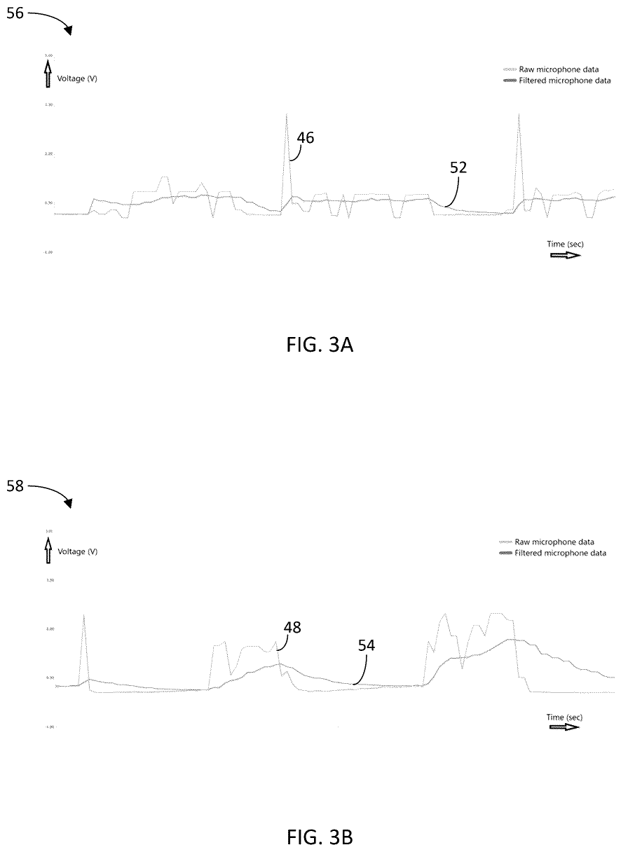

Generally, signals or inputs, received by the microphones 32, 34 (e.g., user voice signals 46 and ambient signals 48) can be provided to the controller 36. In some embodiments, the signals 46, 48 can be filtered before being processed by the controller 36. For example, as shown in FIG. 2, the signals 46, 48 may be filtered using a first order low-pass filter 50 to remove noise and avoid sudden spikes that could affect the feedback of the system 30. These filter signals (e.g., filtered user voice signals 52 and filtered ambient noise signals 54 can then be further analyzed and/or processed by the controller 36. While FIG. 2 illustrates the filter 50 as being separate from the controller 36, it should be noted that, in some embodiments, the filter 50 can be incorporated into the controller 36. Additionally, FIGS. 3A and 3B illustrate example plots 56, 58, of voltage versus time, of raw and filtered signals from the microphones 32, 34. More specifically, FIG. 3A illustrates a plot 56 of signals from the user microphone 32, including a raw user voice signal 46 and a filtered user voice signal 52. FIG. 3B illustrates a plot 58 of signals from the ambient microphone 34, including a raw ambient noise signal 48 and a filtered ambient noise signal 54.

Referring back to FIG. 2, the filtered signals 52, 54 from the microphones 32, 34 can be processed by the controller 36, which can then provide biofeedback to the user via the biofeedback device 38 based on the processed signals. In some embodiments, the biofeedback device 38 can be a tactile feedback device. More specifically, in some embodiments, the biofeedback device 38 can be a vibrotactile feedback device. As such, the controller 36 can provide vibrotactile feedback to the user, e.g., via a control signal 60 to the biofeedback device 38, in response to receiving input signals from the microphones 32, 34.

Additionally, in some embodiments, the biofeedback device 38 can be configured to provide different types of feedback to the user. For example, the biofeedback device 38 can provide a first form of feedback to the user (e.g., based on a first control signal 60 from the controller 36) in response to the user speaking too softly and can provide a second form of feedback to the user (e.g., based on a second control signal 60 from the controller 36), different from the first form of feedback, when the user is speaking too loudly. In further embodiments, additional forms of feedback can be included, such as different levels of feedback based on the user's volume and/or a specific form of feedback when the user is speaking at a normal level. In one specific example, the controller 36 can control the biofeedback device 38 to emit intermittent vibrations when the user is speaking too loudly, prompting the user to reduce their volume, and controller 36 can control the biofeedback device 38 to emit a continuous vibration when the user is speaking too softly, prompting the user to increase their volume.

Accordingly, such tactile feedback can be provided to the user for immediate voice adjustment with minimal mental effort and/or training and without interfering with normal communication patterns (e.g., in comparison to auditory feedback which may interfere with hearing their communication partner or visual feedback which may require the user to focus on a light source rather than making eye contact with conversation participants). That is, by utilizing vibrotactile feedback instead of conventional auditory or visual cues, the system 30 can be seamlessly integrated into conversations, eliminating distractions and helping users maintain their focus on communication.

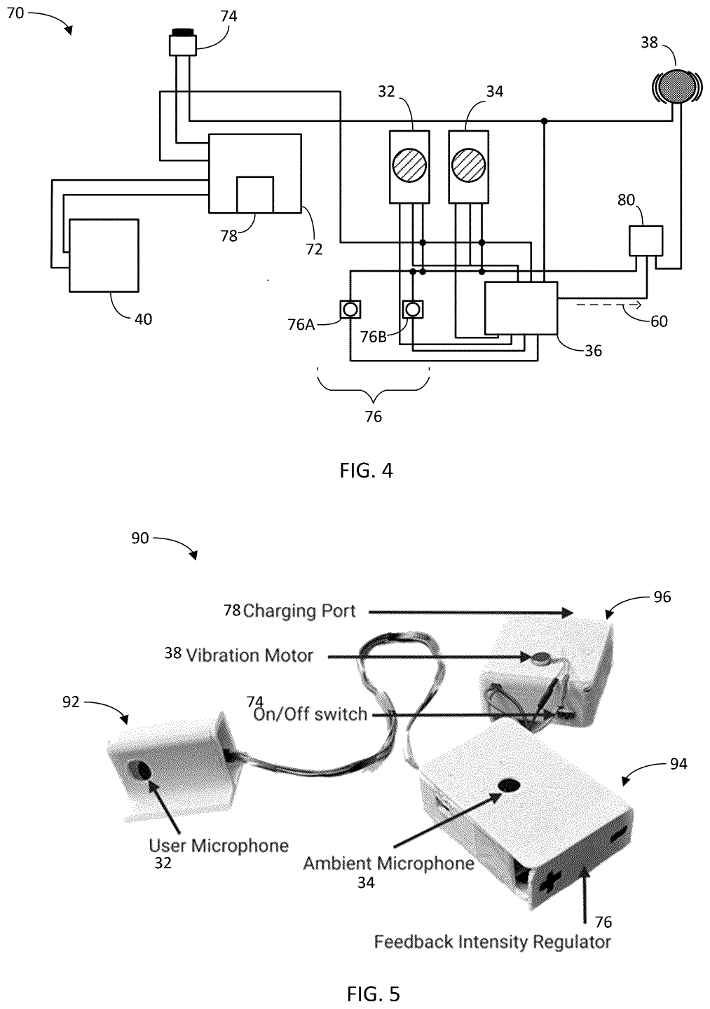

FIG. 4 illustrates an example electronic schematic diagram of a system 70 for speech disorders according to some embodiments. The system 70 of FIG. 4 may be similar to the system 30 of FIG. 2 and, thus, like reference numerals indicate similar components. For example, the system 70 can include the first microphone 32 (e.g., a user microphone), the second microphone 34 (e.g., an ambient microphone), the controller 36, the biofeedback device 38 (e.g., a vibration motor), and the power source 40. The system 70 can further include a power source management system 72, an on/off switch 74, and user inputs 76 (such as user inputs 76A and 76B). It should be noted that, while the components of the system 70 are illustrated as being physically connected to one another via wired connections, in some embodiments, one or more components may be coupled via wireless connections.

Referring still to FIG. 4, in some embodiments, the power source 40 can be a battery. In further embodiments, the power source 40 can be a rechargeable battery. Accordingly, the power source management system 72 can provide the necessary components and connections to facilitate battery charging, such as a charging port 78. Furthermore, the power source management system 72 can be connected between the power source 40 and the other components of the system 70, such as the controller 36 and the biofeedback device 38. As such, power to the system 70 can be controlled by the power source management system 72, for example, via the on/off switch 74. In some embodiments, the on/off switch 74 can be a physical switch, physical button, physical dial, digital button, or other suitable component that can be actuated (e.g., pressed, moved, turned, etc.) by the user to turn on or off the system 70. Accordingly, the rechargeable power source 40 can offer prolonged use and reuse of the system 70, and the power source management system 72 can further prolong the life of the rechargeable power source 40. In one example, the rechargeable power source 40 can have a battery life of about 12 hours on a single charge. And the actual time between charges can be further prolonged by the user switching off the system 70, via the on/off switch 74, when not in use.

While the user can switch the system 70 on and off via the on/off switch 74, the user can further make adjustments to the system 70 via the user inputs 76. For example, in some embodiments, the user inputs 76 can be one or more physical switches, physical buttons, physical dials, digital buttons, or other suitable components that can be actuated (e.g., pressed, moved, turned, etc.) by the user to adjust a vibration intensity of the biofeedback device 38. That is, in one example, the user inputs 76 can be coupled to the controller 36 and, based on signals from the user inputs 76, the controller 36 can adjust a signal to the biofeedback device 38 (e.g., adjust the signal to the vibration motor to increase or decrease vibration intensity). Adjustable inputs will minimize adaptation to the intensity of the biofeedback provided over time. Additionally, users can adjust vibration intensity to suit their preferences. In the example shown in FIG. 4, the user inputs 76 includes two buttons: a first button 76A that, when pressed, increases the vibration intensity of the biofeedback; and a second button 76B that, when pressed, decreases the vibration intensity of the biofeedback. Furthermore, in some examples, as shown in FIG. 4, the controller 36 can transmit the control signals 60 (as shown and described above with reference to FIG. 2) to the biofeedback device 38 via a transistor 80. More specifically, the controller 36 can selectively apply power from the power source 40 to the biofeedback device 38 by applying the control signal 60 to the transistor 80.

FIG. 5 illustrates another example system 90 for speech disorders according to some embodiments. For example, the system 90 of FIG. 5 can incorporate the electronic components of FIG. 4 in corresponding housings, such as 3D printed cases. As such, the system 90 of FIG. 5 may be similar to the system 30 of FIG. 2 and the system 70 of FIG. 4 and, thus, like reference numerals indicate similar components. For example, the system 90 can include a first housing 92 including the first microphone 32 (e.g., a user microphone), a second housing 94 including the second microphone 34 (e.g., an ambient microphone), the controller 36 (hidden from the view shown in FIG. 5), and the user inputs 76, and a third housing 96 including the biofeedback device 38 (e.g., a vibration motor), the power source 40 (hidden from the view shown in FIG. 5), the power source management system 72 (hidden from the view shown in FIG. 5), and the on/off switch 74.

It should be noted that, while the components of the system 90 are illustrated as being physically connected to one another via wired connections, in some embodiments, one or more components may be coupled via wireless connections. Furthermore, in some embodiments, the wired connections may be removable, allowing the housings 92, 94, 96 to be disconnected from each other and reconnected. In this manner, the third housing 96 could be disconnected from the other housings 92, 94 to allow for battery charging while not disturbing the other housings 92, 94. Additionally, while the components are shown and described herein as being contained within three separate housings 92, 94, 96, in some embodiments, more or fewer housings may be used and the components may be in different housings than what is specifically described herein. In some embodiments, however, keeping the biofeedback device 38, the user microphone 32, and the ambient microphone 34 in separate housings 96, 92, 94 can be beneficial so that the user microphone 32 and the ambient microphone 34 avoid interference from the vibration motor of the biofeedback device 38.

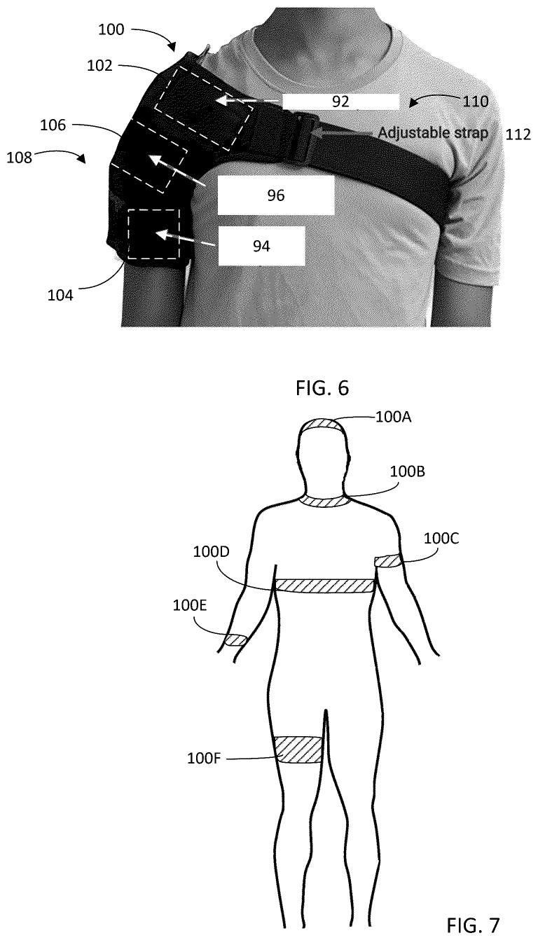

In some embodiments, the system 90 can be incorporated into a wearable pack that can be held by the user. In further embodiments, the system 90 can be incorporated into a wearable garment. For example, FIG. 6 illustrates a wearable garment in the form of a shoulder brace 100, where, according to some embodiments, the housings 92, 94, 96 of the system 90 can be placed inside the shoulder brace 100. In other words, the system 90 and, specifically, components of the system 90 can be “incorporated into,” e.g., coupled to, placed within, and/or attached to the shoulder brace 100.

More specifically, in some embodiments, the shoulder brace 100 can include pockets 102, 104, 106 to receive respective housings 92, 94, 96 of the system 90 of FIG. 5 and accommodate the associated wired connections. Such a design can make the housings 92, 94, 96 hidden from outside view (and, thus, not viewable in FIG. 6) when wearing the shoulder brace 100. Furthermore, as the shoulder brace 100 could be worn under regular attire, the entire system 90 can be completely hidden from view when in use. This design, therefore, allows users to employ the system 90 without drawing attention, thus enhancing user privacy, confidence, and promoting consistent use across various social contexts.

In some embodiments, the pockets can allow for one or more of the housings 92, 94, 96 to be removed. Thus, in such embodiments, the user can remove the system 90 from the shoulder brace 100, for example, during washing or during recharging. In other embodiments, one or more of the housings 92, 94, 96 can be permanently affixed to the shoulder brace 100 within a respective pocket 102, 104, 106. Alternatively, in some embodiments, one or more of the housings 92, 94, 96, or one or more individual components of the system 90, can be affixed to an outer surface of the shoulder brace 100, e.g., rather than within a respective pocket 102, 104, 106.

Generally, in some embodiments, the shoulder brace 100 can be lightweight, flexible, and adjustable to accommodate all body types comfortably and allow users to comfortably use the system 90 in day-to-day activities. As shown in FIG. 6, the shoulder brace 100 can include an arm portion 108 that fits over an upper arm of a user and a strap 110 adapted to fit around the user's chest to secure the shoulder brace 100 to the user. In some embodiments, the strap 110 can be adjustable to better secure the shoulder brace 100 to the user tight enough for the user to feel the tactile feedback from the biofeedback device 38. For example, the strap 110 can be adjustable via a buckle 112 or other suitable components.

Regarding housing placement, in some embodiments, as shown in FIG. 6, the system 90 can generally fit into pockets 102, 104, 106 within the arm portion 108 of the shoulder brace 100. For example, the first housing 92, containing the first microphone 32 (as shown in FIG. 5), can be positioned in a first pocket 102 near the user's collarbone, allowing the first microphone 32 to pick up the user's voice. The second housing 94, containing the second microphone 34 (as shown in FIG. 5), can be positioned in a second pocket 104 closer to the user's elbow (e.g., further from the user's mouth than the first housing 92), allowing the second microphone 34 to pick up ambient noise. The third housing 96, containing the biofeedback device 38 (as shown in FIG. 5) and power source 40 (shown in FIG. 4), can be positioned in a third pocket 106 near a “middle” of the arm portion 108, adjacent the user's deltoid. In this manner, the biofeedback device 38 can be fitted snuggly against the user's arm with minimal variance regardless of the user's arm position and movement. It should be noted, however, that while the housings 92, 94, 96 are illustrated and described herein as being in particular positions along the shoulder brace 100, other positions of the housings 92, 94, 96 and/or individual components of the system 90 may be contemplated in some embodiments.

Accordingly, a user may don the system 90 via the shoulder brace 100. Once the system 90 is turned on (e.g., by actuating the on/off switch 74), the ambient microphone 34 can start capturing the ambient noise. When the user starts speaking, the captured ambient noise is used for comparison with the user's voice level, as captured by the user microphone 32, and the system 90 provides tactile feedback via the biofeedback device 38. That is, the system 90 can provide continuous conversation vibrotactile feedback without external intervention (i.e., without feedback from a conversation partner) that is based on the user's voice and ambient noise. It should be noted that, while the wearable garment is shown and described herein as a shoulder brace 100, other types of wearable garments may be contemplated in some embodiments, such as chest straps, arm sleeves, arm bands, leg sleeves, leg bands, headbands, shirts, shorts, pants, neckbands, rings, patches, watches, bracelets, etc. For example, FIG. 7 illustrates example locations of wearable garments, such as a headband 100A, a neckband 100B, an armband 100C, a chest strap 100D, a bracelet 100E, and a leg band 100F, that may incorporate some or all components of the system 90 (or the systems 30, 70). Other locations or sizes of wearable garments, other than what is shown in FIGS. 6 and 7, may be used in some embodiments.

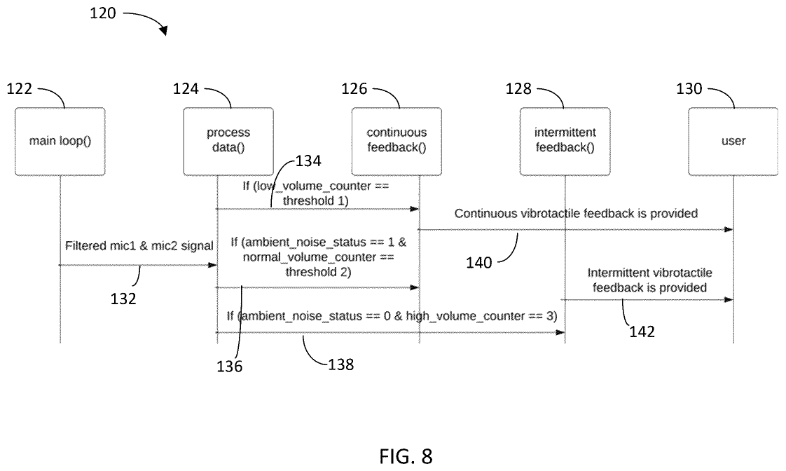

FIG. 8 illustrates a sequence flow diagram 120 of feedback logic of the controller 36 (which is illustrated in FIGS. 2 and 4), according to some embodiments, as a method for providing continuous and intermittent vibrotactile feedback. For example, this feedback logic can be stored as computer-readable instructions in the memory 44 of the controller 36 and executed by the processor 42 (shown in FIG. 2). Alternatively, in other examples, this feedback logic may be executed via logic circuits of the controller 36. Throughout the following description, reference may be made to components of the controller 36 or associated systems 30, 70, 90 illustrated in FIGS. 2, 4, and 5.

As shown in FIG. 8, the diagram 120 can include a main loop block 122, a process data block 124, a continuous feedback block 126, an intermittent feedback block 128, and a user block 130. The main loop block 122 indicates the beginning of the flow sequence when inputs from both of the microphones 32, 34 are received. The process data block 124 indicates data processing performed by the controller 36. The continuous feedback block 126 indicates when continuous tactile feedback (e.g., a first form of feedback) should be output to the user. The intermittent feedback block 128 indicates when intermittent tactile feedback (e.g., a second form of feedback) should be output to the user. The user block 130 indicates when feedback outputs are provided to the user, e.g., in the form of tactile (vibrational) feedback. It should be noted that, while the feedback logic and separate blocks are shown and described herein in a particular order to help explain system operations, in some embodiments, the controller 36 may not include separate modules corresponding to each block of FIG. 8 and/or may include modules that combine one or more blocks or functions of FIG. 8.

Referring still to FIG. 8, the main loop block 122 indicates the beginning of the flow sequence when inputs from both the microphones 32, 34 are received. Filtered signals from the microphones 32, 34 are provided to the process data block 124 via line 132. At the process data block 124, the controller 36 processes the filtered microphone signals to determine a user's vocal state (e.g., 1, 2, or 3, corresponding to low, normal, or high, respectively) and an ambient state (e.g., 1 or 2, corresponding to noise and no noise, respectively). If the user's vocal state is determined to be low (vocal state=1), in relation to the determined ambient state (ambient state=1 or 2), the process proceeds to the continuous feedback block 126 at line 134. If the user's vocal state is determined to be normal (vocal state=2), but ambient state is determined to be noisy (ambient state=1), the process proceeds to the continuous feedback block 126 at line 136. If the user's vocal state is determined to be high (vocal state=3) and ambient state is determined to be not noisy (ambient state=2), the process proceeds to the intermittent feedback block 128 at line 138.

When signals are received at the continuous feedback block 126 (e.g., from lines 134 or 136 in FIG. 8), this indicates that the user may be speaking too quietly. Therefore, continuous vibrotactile feedback is provided to the user (at user block 130) via line 140 to indicate to the user to speak louder. When signals are received at the intermittent feedback block 128 (e.g., from line 138 in FIG. 8), this indicates that the user may be speaking too loudly. Therefore,-intermittent vibrotactile feedback is provided to the user (at user block 130) via line 142 to indicate to the user to speak quieter.

Additionally, it should be noted that other states may exist that are not shown in FIG. 8. Example situations can include where the user's vocal state is determined to be normal (vocal state=2), and ambient state is determined to be not noisy (ambient state=2), or the user's vocal state is determined to be high (vocal state=3) and ambient state is determined to be noisy (ambient state=1). In these situations, no feedback may be output via the biofeedback device 38, indicating to the user that their vocal intensity is satisfactory for the current environment. That is, the absence of tactile feedback is, in itself, another form of feedback to the user indicating that their vocal level is satisfactory. However, in some embodiments, another different form of tactile feedback may be incorporated into the system 90 when such states are determined.

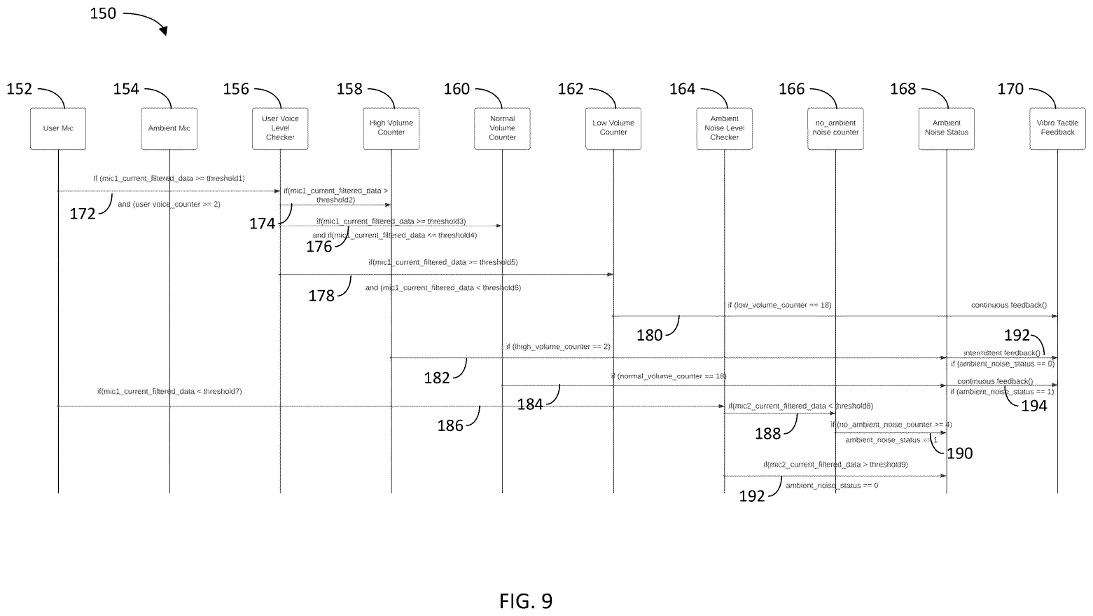

Additionally, FIG. 9 illustrates another example sequence flow diagram 150 of feedback logic of the controller 36, according to some embodiments, as a method for providing continuous and intermittent vibrotactile feedback. For example, this feedback logic can be stored as computer-readable instructions in the memory 44 of the controller 36 and executed by the processor 42 (shown in FIG. 2). Alternatively, in other examples, this feedback logic may be executed via logic circuits of the controller 36. Throughout the following description, reference may be made to components of the controller 36 or associated systems 30, 70, 90 illustrated in FIGS. 2, 4, and 5. As shown in FIG. 9, the flow diagram 150 can include a user microphone block 152, an ambient microphone block 154, a user voice level checker 156, a high volume counter 158, a normal volume counter 160, a low volume counter 162, an ambient noise level checker 164, a no-ambient noise counter 166, an ambient noise status block 168, and a vibro-tactile feedback block 170. For example, the vibro-tactile feedback block 170 can indicate when feedback outputs are provided to the user, e.g., in the form of tactile (vibrational) feedback.

Referring still to FIG. 9, if input from the user microphone block 152, such as the filtered user voice signals 52 shown in FIG. 2, is greater than or equal to a threshold, and a user voice counter is greater than or equal to a threshold, the sequence proceeds to the user voice level checker 156 (line 172). At the user voice level checker 156, if the filtered user voice signals 52 are greater than a first threshold, the sequence proceeds to the high volume counter 158, e.g., the sequence adds a count to the high volume counter 158 (line 174). If the filtered user voice signals 52 are greater than or equal to a second threshold and less than or equal to a third threshold, the sequence proceeds to the normal volume counter 160, e.g., the sequence adds a count to the normal volume counter 160 (line 176). If the filtered user voice signals 52 are greater than or equal to a fourth threshold and less than or equal to a fifth threshold, the sequence proceeds to the low volume counter 162, e.g., the sequence adds a count to the low volume counter 162 (line 178).

If the low volume counter 162 is equal to a predetermined number, such as 18 in this example, the sequence proceeds to the vibro-tactile feedback block 170 to provide continuous vibro-tactile feedback (line 180), e.g., indicating to the wearer that they are speaking too quietly. In other words, if the wearer is speaking too softly, regardless of ambient noise, feedback may be given to the wearer via the system 30, 70, 90 indicating that the wearer may be speaking too softly for their present environment. Counts from the high volume counter 158 or the normal volume counter 160 may also result in the vibro-tactile feedback block 170 providing vibro-tactile feedback, though such feedback may be further based on ambient noise levels.

More specifically, if input from the user microphone block 152 (e.g., the filtered user voice signals 52) is less than a threshold, the sequence proceeds to the ambient noise level checker 164 (line 186). If, at the ambient noise level checker 164, input from the ambient microphone block 154, such as the filtered ambient noise signals 54 shown in FIG. 2, is less than a threshold, the sequence proceeds to the no-ambient noise counter 166, e.g., the sequence adds a count to the no-ambient noise counter 166 (line 188). And, if the no-ambient noise counter is greater than or equal to a threshold, such as four in this example, the sequence proceeds to the ambient noise status block 168 indicating ambient noise status as 1 (line 190). However, if input from the ambient microphone block 154 (e.g., the filtered ambient noise signals 54) is greater than a threshold, the sequence proceeds to the ambient noise status block 168 indicating ambient noise status as 0 (line 192).

Looking back to the high volume counter 158, if the high volume counter 158 is equal to a predetermined number, such as two in this example, indicating that the user is speaking loudly, the sequence proceeds to the ambient noise status block 168 (line 182). If the ambient noise status block 168 is at 0, indicating low ambient noise, the sequence proceeds to the vibro-tactile feedback block 170 to provide intermittent vibro-tactile feedback (line 192), e.g., indicating to the wearer that they are speaking too loudly. In other words, if the wearer is speaking loudly and there is little ambient noise, feedback may be given to the wearer via the system 30, 70, 90 indicating that the wearer may be speaking too loudly for their present environment.

Looking back to the normal volume counter 160, if the normal volume counter 160 is equal to a predetermined number, such as 18 in this example, the sequence proceeds to the ambient noise status block 168 (line 184). If the ambient noise status block 168 is at 1, indicating high ambient noise, the sequence proceeds to the vibro-tactile feedback block 170 to provide continuous vibro-tactile feedback (line 194), e.g., indicating to the wearer that they are speaking too softly. In other words, if the wearer is speaking at a normal level but there is high ambient noise, feedback may be given to the wearer via the system 90 indicating that the wearer may be speaking too softly for their present environment.

Accordingly, in contrast to other options currently available, the system 90 (or the systems 30, 70) of some embodiments offers feedback for both high and low vocal intensities. Further, the system 90 assesses both the user's vocal intensity and the ambient noise levels. This comprehensive approach ensures that users receive appropriate feedback regardless of their vocal output and dynamically adjusts feedback in response to changing noise conditions. Furthermore, by taking into account the surrounding environment, the system 90 can assist the user to achieve proper vocal intensity across a variety of settings. That is, when the user speaks at a low volume, relative to ambient noise, continuous vibrotactile feedback will be generated. When there is no ambient noise and the user converses in a normal voice, no feedback is provided, but if the user is conversing in a normal voice in the presence of ambient noise, continuous feedback will be provided. If the user speaks at a high volume in the absence of ambient noise, intermittent vibrotactile feedback will be provided, prompting the user to reduce their volume, but if ambient noise is present and the user speaks at a high volume, feedback is not provided.

In light of the above, some embodiments provide a biofeedback system and method catering to individuals challenged by speech disorders and/or communication difficulties, e.g., originating from neurodegenerative conditions. These individuals often grapple with the ability to speak clearly, which may be because they struggle to perceive and adjust intensity independently. However, a remarkable transformation occurs when they receive external feedback, such as with the present system. The system of some embodiments aims to empower these individuals to engage in more natural conversations by offering real-time guidance in modulating their vocal intensity. Using vibrotactile feedback, the system assists users in adjusting their vocal intensity without external human intervention. The system incorporates strategically placed microphones that collectively capture the user's voice and ambient sounds, which are filtered through a low-pass filter, and the filtered signals are interpreted by a responsive controller that generates tailored vibrotactile feedback through a vibration motor. By providing instant guidance on adapting vocal intensity to ambient noise, the system enables comfortable conversations without external intervention, thus enhancing the user's independence and confidence. Its discreet design ensures seamless integration into daily life, enhancing communication and fostering inclusivity for those with developmental, acquired, or neurodegenerative speech disorders. Furthermore, this system can be used for treatment of voice disorders associated with neurodegenerative disorders and other acquired brain injuries. As patients work to adapt their vocal intensity in clinic-based therapy sessions and eventually in real-life settings, this system provides tactile feedback about their success with achieving their targeted volume. It is important for patients to be able to self-monitor their speaking volume and adjust it as needed, without relying on cues from the therapist.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

### Numerated Embodiments

Embodiment 1: A feedback system for speech modulation of a user, the feedback system comprising:
a first microphone to capture the user's voice;
a second microphone to capture ambient noise;
a controller to process inputs from the first microphone and the second microphone corresponding to the user's voice and the ambient noise, respectively, and provide a control signal based on the inputs; and
a biofeedback device to receive the control signal from the controller to provide tactile feedback to the user, the biofeedback device to provide:
   a first form of tactile feedback indicating the user is speaking too softly; and
   a second form of tactile feedback indicating the user is speaking too loudly.

Embodiment 2: The feedback system of embodiment 1, wherein the first form of tactile feedback is continuous vibrotactile feedback and the second form of tactile feedback is intermittent vibrotactile feedback.

Embodiment 3: The feedback system of embodiment 1 or 2, wherein the first microphone is an adjustable gain microphone and the second microphone is an auto-gain microphone.

Embodiment 4: The feedback system of any one of embodiments 1-3, further comprising a low-pass filter to filter the inputs from the first microphone and the second microphone before they are processed by the controller.

Embodiment 5: The feedback system of any one of embodiments 1-4, further comprising a user input to receive input from the user to adjust an intensity of the tactile feedback.

Embodiment 6: The feedback system of any one of embodiments 1-5, further comprising a rechargeable power source that provides power to the controller via a power source management system and an on/off switch.

Embodiment 7: A feedback system for speech modulation of a user, the feedback system comprising:
a wearable garment;
a first microphone, incorporated into the wearable garment, to capture the user's voice;
a second microphone, incorporated into the wearable garment, to capture ambient noise;
a controller to process inputs from the first microphone and the second microphone corresponding to the user's voice and the ambient noise, respectively; and
a biofeedback device, incorporated into the wearable garment, controlled by the controller to provide tactile feedback to the user based on the processed inputs.

Embodiment 8: The feedback system of embodiment 7, wherein the biofeedback device is to provide:
a first form of tactile feedback indicating the user is speaking too softly; and
a second form of tactile feedback indicating the user is speaking too loudly.

Embodiment 9: The feedback system of embodiment 7 or 8, wherein the wearable garment is a shoulder brace.

Embodiment 10: The feedback system of embodiment 9, wherein the shoulder brace comprises:
a first pocket to receive a first housing comprising the first microphone;
a second pocket to receive a second housing comprising the second microphone; and
a third pocket to receive a third housing comprising the biofeedback device.

Embodiment 11: The feedback system of embodiment 10, wherein the first housing, the second housing, and the third housing are removable from the first pocket, the second pocket, and the third pocket, respectively.

Embodiment 12: The feedback system of embodiment 10 or 11, wherein the first housing, the second housing, and the third housing are connected via wired connections.

Embodiment 13: The feedback system of embodiment 12, wherein the wired connections between the first housing, the second housing, and the third housing are configured to be disconnected from each other.

Embodiment 14: The feedback system of embodiment 7 or 8, wherein the wearable garment includes one of a chest strap, an arm sleeve, an arm band, a leg sleeve, a leg band, a headband, a shirt, shorts, pants, a neckband, a ring, a patch, a watch, and a bracelet.

Embodiment 15: A method of providing feedback to a user for speech modulation, the method comprising:
acquiring voice signals corresponding to a voice of the user;
acquiring ambient signals corresponding to an ambient environment;
processing the voice signals to determine a user's vocal state;
processing the ambient signals to determine an ambient state; and
providing tactile feedback to the user based on the user's vocal state relative to the ambient state.

Embodiment 16: The method of embodiment 15, wherein providing tactile feedback to the user based on the user's vocal state relative to the ambient state includes:

providing a first form of tactile feedback indicating the user is speaking too softly; and providing a second form of tactile feedback indicating the user is speaking too loudly.

Embodiment 17: The method of embodiment 15 or 16, wherein the user's vocal state is selected from a list comprising low, normal, and high, and the ambient state is selected from a list comprising noisy and not noisy, and further comprising determining that the user is speaking too softly when the user's vocal state is low.

Embodiment 18: The method of any one of embodiments 15-17, further determining that the user is speaking too softly when the user's vocal state is normal and the ambient state is noisy.

Embodiment 19: The method of any one of embodiments 15-18, wherein the user's vocal state is selected from a list comprising low, normal, and high, and the ambient state is selected from a list comprising noisy and not noisy; and further comprising determining that the user is speaking too loudly when the user's vocal state is high, and the ambient state is not noisy.

Embodiment 20: The method of any one of embodiments 15-18, wherein:
providing the first form of tactile feedback indicating the user is speaking too softly includes providing continuous vibrotactile feedback; and
providing the second form of tactile feedback indicating the user is speaking too loudly includes providing intermittent vibrotactile feedback.

1. A feedback system for speech modulation of a user, the feedback system comprising:
a first microphone to capture the user's voice;
a second microphone to capture ambient noise;
a controller to process inputs from the first microphone and the second microphone corresponding to the user's voice and the ambient noise, respectively, and provide a control signal based on the inputs; and
a biofeedback device to receive the control signal from the controller to provide tactile feedback to the user, the biofeedback device to provide:
a first form of tactile feedback indicating the user is speaking too softly; and
a second form of tactile feedback indicating the user is speaking too loudly.

2. The feedback system of claim 1, wherein the first form of tactile feedback is continuous vibrotactile feedback and the second form of tactile feedback is intermittent vibrotactile feedback.

3. The feedback system of claim 1, wherein the first microphone is an adjustable gain microphone and the second microphone is an auto-gain microphone.

4. The feedback system of claim 1, further comprising a low-pass filter to filter the inputs from the first microphone and the second microphone before they are processed by the controller.

5. The feedback system of claim 1, further comprising a user input to receive input from the user to adjust an intensity of the tactile feedback.

6. The feedback system of claim 1, further comprising a rechargeable power source that provides power to the controller via a power source management system and an on/off switch.

7. A feedback system for speech modulation of a user, the feedback system comprising:
a wearable garment;
a first microphone, incorporated into the wearable garment, to capture the user's voice;
a second microphone, incorporated into the wearable garment, to capture ambient noise;
a controller to process inputs from the first microphone and the second microphone corresponding to the user's voice and the ambient noise, respectively; and
a biofeedback device, incorporated into the wearable garment, controlled by the controller to provide tactile feedback to the user based on the processed inputs.

8. The feedback system of claim 7, wherein the biofeedback device is to provide:
a first form of tactile feedback indicating the user is speaking too softly; and
a second form of tactile feedback indicating the user is speaking too loudly.

9. The feedback system of claim 7, wherein the wearable garment is a shoulder brace.

10. The feedback system of claim 9, wherein the shoulder brace comprises:
a first pocket to receive a first housing comprising the first microphone;
a second pocket to receive a second housing comprising the second microphone; and
a third pocket to receive a third housing comprising the biofeedback device.

11. The feedback system of claim 10, wherein the first housing, the second housing, and the third housing are removable from the first pocket, the second pocket, and the third pocket, respectively.

12. The feedback system of claim 10, wherein the first housing, the second housing, and the third housing are connected via wired connections.

13. The feedback system of claim 12, wherein the wired connections between the first housing, the second housing, and the third housing are configured to be disconnected from each other.

14. The feedback system of claim 7, wherein the wearable garment includes one of a chest strap, an arm sleeve, an arm band, a leg sleeve, a leg band, a headband, a shirt, shorts, pants, a neckband, a ring, a patch, a watch, and a bracelet.

15. A method of providing feedback to a user for speech modulation, the method comprising:
acquiring voice signals corresponding to a voice of the user;
acquiring ambient signals corresponding to an ambient environment;
processing the voice signals to determine a user's vocal state;
processing the ambient signals to determine an ambient state; and
providing tactile feedback to the user based on the user's vocal state relative to the ambient state.

16. The method of claim 15, wherein providing tactile feedback to the user based on the user's vocal state relative to the ambient state includes:
providing a first form of tactile feedback indicating the user is speaking too softly; and
providing a second form of tactile feedback indicating the user is speaking too loudly.

17. The method of claim 16, wherein the user's vocal state is selected from a list comprising low, normal, and high, and the ambient state is selected from a list comprising noisy and not noisy; and further comprising determining that the user is speaking too softly when the user's vocal state is low.

18. The method of claim 17, further comprising determining that the user is speaking too softly when the user's vocal state is normal and the ambient state is noisy.

19. The method of claim 16, wherein the user's vocal state is selected from a list comprising low, normal, and high, and the ambient state is selected from a list comprising noisy and not noisy; and further comprising determining that the user is speaking too loudly when the user's vocal state is high, and the ambient state is not noisy.

20. The method of claim 16, wherein:
providing the first form of tactile feedback indicating the user is speaking too softly includes providing continuous vibrotactile feedback; and
providing the second form of tactile feedback indicating the user is speaking too loudly includes providing intermittent vibrotactile feedback.
